(19) United States
(12) Patent Application Publication
(10) Pub. No.: US 2007/0276218 A1
(43) Pub. Date: Nov. 29, 2007

(54) Magnetic markers for position sensing

(76) Inventor: Benjamin Yellen, Durham, NC (US)

Correspondence Address: Steven E. Bach;Attorney at Law, 10 Roberts Road, Newtown Square, PA, 19073, US

(21) Appl. No.: 11/417,601

(22) Filed: May 4, 2006

(51) Int. Cl. A61B 5/05

(52) U.S. Cl. 600/409

(57) ABSTRACT

An imaging system is provided for locating implants within a patient, and for determining the spatial relationship and orientation between components of an implant within a patient. Magnets are fixed to an implant in pre-determined locations, the magnets having pre-determined quantity, magnetic field map, and moment orientations. An array of sensors is arranged around the implant. The locations of the magnets are estimated by iteratively solving for the position vector coordinates using multi-poles approximating the pre-determined quantity, magnetic field map, and moment orientations of the magnets and the magnetic field measurements for each sensor. The positions and orientations of the implant or components of the implant are inferred from the estimated locations of the magnets.

### FIELD OF THE INVENTION

The invention is related to the field of magnetic imaging and more particularly to the use of magnetic imaging for position and orientation sensing of implanted devices.

### BACKGROUND

Typical imaging systems for probing structures inside the body include Magnetic Resonance Imaging (MRI) and Computerized Tomography (CT). MRI and CT provide high resolution images which are important for many imaging applications including the use of imaging to place implants and to monitor implant wear, for example. However, MRI and CT are not compatible with implants that contain metallic components. In MRI systems, metallic components respond to RF magnetic fields by producing eddy currents and a resulting distortion in the observed image. In CT systems, metallic components interfere with image acquisition because X-rays cannot effectively penetrate dense metals.

Over the last few decades, experimental and computational techniques have been developed for mapping low frequency magnetic fields emerging from biological sources, such as electrical fluctuations in the heart, MagnetoCardiography (MCG), or brain, MagnetoEncephelography (MEG), or magnetic dust particles embedded in the lungs, MagnetoPneumography (MPG). These imaging systems are based on mapping slowly varying or static magnetic fields, and are relatively unaffected by the presence of non-magnetic metallic components. However, these imaging systems have suffered from relatively poor resolution, because the source parameters (e.g., locations, orientations, and magnitudes) can vary widely and little a priori information is available for their determination. Moreover, the magnetic fields produced by these sources are inherently weak. They can typically be detected only by expensive SQUID sensors, and with poor resolution (i.e., about 1 cm).

### SUMMARY

According to an exemplary embodiment of the invention, an imaging system is provided for locating implants within a patient, and for determining the spatial relationship and orientation between components of an implant within a patient. Magnets are fixed to an implant, the magnets having pre-determined quantity, magnetic field map, and moment orientations. An array of sensors is arranged around the implant. The locations of the magnets are estimated by iteratively solving for the position vector coordinates using the pre-determined quantity, magnetic field map, and moment orientations of the magnets and the magnetic field measurements for each sensor. The positions and orientations of the implant and/or components of the implant are inferred from the estimated locations of the magnets.

### BRIEF DESCRIPTION OF THE ACCOMPANYING DRAWINGS

The invention will be described in greater detail below with reference to the accompanying drawings, of which:

FIG. 1 shows an imaging system according to an exemplary embodiment of the invention;

FIG. 2 shows an implant with magnets fixed thereto according to an exemplary embodiment of the invention; and

FIGS. 3 and 4 and show details of components of the implant with magnets of FIG. 2.

### DETAILED DESCRIPTION

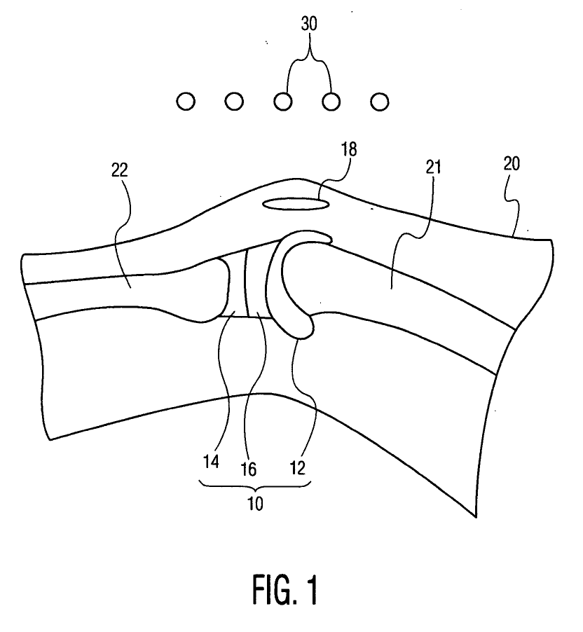

According to an exemplary embodiment of the invention, an imaging system is configured to determine the position of an implant 10 within a patient's body 20. As shown in FIG. 1, an array of magnetic sensors 30 is disposed in a pattern around the implant 10 (as well as the body part where the implant is located). The magnetic sensors 30 may be arranged in a plane directly above or below the implant, as shown in FIG. 1. Alternatively, the magnetic sensors 30 may be arranged in a magnetic brace (not shown) in the shape of a cylinder surrounding the implant. The array of magnetic sensors 30 may also be formed by moving one or more sensors over a known distance and direction and measuring the magnetic field at each location. The magnetic sensor apparatus may also contain a system for swiping the sensor array across the patient's body part in order to generate more imaging data.

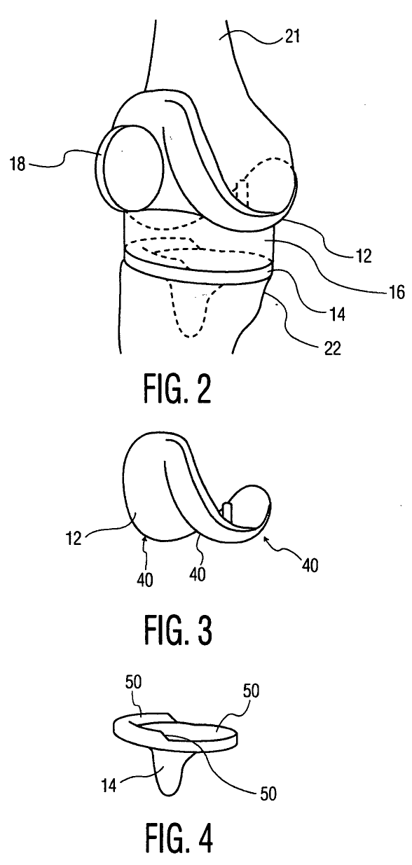

The magnetic sensors 30 may be any of a variety of commercially available magnetic field sensors, for example arrays of Giant MagnetoResistive sensors (GMR) and Magetic Tunneling Junction sensors (MTJ) are available from Micro Magnetics, Inc. of Fall River, MA and NVE Corporation of Eden Prarie, Minn. While expensive and highly sensitive SQUID sensors may be used, the present invention may advantageously be practiced with less expensive sensors, because the magnets 40, 50 (see FIGS. 3 and 4) fixed to the implants 10 produce a sufficiently strong field signal that can be detected by less sensitive sensors.

Inverse imaging solutions (where measurements made exterior to an enclosed region are used to deduce properties of the hidden interior) tend to be computationally expensive and do not provide good resolution, especially when little a priori information is known. In transient field systems such as MCG, MEG, and MPG little a priori information is known about the nature and location of the field source.

In the present invention, an implant 10 is designed to maximize the a priori information available. In particular, a known number of magnets 40, 50 are used in the implant 10. Moreover, the magnets 40, 50 are intentionally designed to produce known fields, such as for example, characteristically dipolar and quadrupolar fields. Other source parameters, such as moment orientation, may be controlled prior to image capture by magnetizing the magnets 40, 50 in a preferred direction using an external apparatus.

An exemplary implant 10 is shown in FIG. 2. This implant 10 comprises a knee joint having a femoral liner 12 (attached to the end of the femor), a tibial tray 14 (attached to the end of the tibia 22), a bearing 16 (rotatably fixed to the tibial tray and in sliding engagement with the femoral liner), and a patella button 18. After implantation, it is desirable to determine the positioning and orientation of the liner 12 and tray 14 relative to each other to determine the condition and operation of the implant 10. For example, it is desirable to monitor the wear of the bearing 16 which typically comprises a material that is susceptible to wear, such as polyethylene. This wear can be calculated from the change in the distance between the femoral liner 12 and the tibial tray 14. Similarly, it is desirable to measure the alignment and positioning of the femoral liner 12 relative to the tibial tray 14 and the path of motion of the femoral liner 12 relative to the tibial tray 14.

The femoral liner 12 and tibial tray 14 are each characterized in three dimensions by three magnets 40, 50. Three magnets 40 are affixed to the femoral liner 12 at predetermined locations. The magnets 40 may be affixed to the surface of the femoral liner in a non-contact area or may be embedded below the surface of the femoral liner 12. As will be understood by those skilled in the art, the femoral liner 12, having a known size and shape can be located and oriented in three dimensions from the three known points on the femoral liner 12 located by the three magnets 40. Similarly, the tibial tray 14 has three magnets 50 affixed to it at predetermined locations. The location and orientation of the tibial tray 14 can be determined from the three-dimensional positions of these three magnets 50. The relative positions and orientations of the tibial tray 14 and femoral liner 12 can also be determined from these six points, located by the magnets 40, 50.

The three dimensional positions of the magnets 40, 50 are determined by measuring the magnetic field at each magnetic sensor 30 in an array of magnetic sensors, and comparing these measured fields to predicted fields at each sensor 30 given a hypothetical set of locations for the magnets 40, 50 relative to the array of sensors. The imaging process consists of shifting the positions of the hypothetical magnets 40, 50 around on a three dimensional grid, and comparing the predicted field to the observed field data. The output of the process is the spatial locations of hypothetical magnets 40, 50 with the pre-determined multipole coefficients having a predicted field which best matches the observed data. A common method for comparing the predicted and observed data is through the least squares approach, where the algorithm searches for a minimum error between the observed and predicted field signal. Such iterative processes are continued until an acceptable positional solution (i.e., acceptable error) is obtained or until a sufficient area has been tested at a sufficient interval.

It is preferable to use iterative search algorithms for systems which are highly constrained (i.e. have a large amount of a priori information available such as the positions, locations, and numbers of magnets). Moreover, specially designed magnets can produce highly consistent magnetic field maps, which can be represented with fewer sets of parameters. For example, in the present invention the magnets are small enough to be represented by multipole expansion analysis, consisting of dipoles, quadrupoles, octopoles, and potentially higher order terms. The dipole moment can be represented uniquely by only two parameters, and the quadruple moment by five parameters. In order to minimize the number of input parameters required for estimation, it is preferable to employ systems where the octopole and higher order terms are negligible. Otherwise, the number of input parameters required for representing each magnet becomes unmanageable for an efficient searching routine. For example, the number of input parameters to describe the field of six magnets using only dipole moment parameters is only 12, whereas when quadrupole moments are included the number of input parameters becomes 42.

The purpose of the magnetization parameters is to provide a better hypothetical model for the field produced by the magnets, such that the positions of each magnet can be determined more precisely. Even if the magnetizations of each element are exactly known, the computational overhead can still vary tremendously depending on the region of space that the algorithm must search through. If you consider the 3-dimensional region of space to be divided into 1,000,000 points (i.e. a 100×100×100 grid), it requires 10ˆ6 iterations to locate a single magnet existing in this region of space. The number of iterations required to locate the positions of n independent magnets in the system becomes 10ˆ6n, which can quickly become an unmanageable number for most computer systems. For a system containing six of such magnets, it would require 10ˆ36 iterations, which is unmanageable except with sophisticated networks of computers. However, it is possible to reduce the number of iterations if some of the magnets are fixed with respect to one another. Groups of magnets which all have fixed relation orientation can be described with fewer output parameters and thus requires fewer iterations. For a system containing six magnets, grouped into two systems of three magnets, the search algorithm requires only 10ˆ24 iterations to locate all components in the aforementioned 1,000,000 point grid. Thus, the imaging system provided herein has the capability to search for the relative 3-dimensional orientation of one component in an orthopedic implant with respect to another by searching for 12 positional and orientation components contained within two groups of three magnets.

It should be noted that the number of iterations shown above is only meant to indicate general trends. The searching algorithm may be optimized by searching through smaller regions of space. This can be accomplished by combining the imaging system with ultrasound or CT based imaging techniques. More efficient computational algorithms can also be derived than the basic one shown above. However, the general concept used to locate two mobile supports of an implant are adequately represented above.

The combination of a priori information available in the foregoing constrained system, including a priori knowledge of: (1) the number of magnets, (2) the magnetic field map produced by each magnet (easily calculated using the geometry, size, and composition of the magnet or easily measured), and (3) moment orientation of each magnet, allows for a magnetically encoded implant 10 to be imaged at multiple locations, potentially with sub-millimeter resolution. In an exemplary embodiment, an orthopedic implant, such as a knee joint may be visualized, and micro-motion of the joint may be studied.

In order to generate a magnetic field sufficiently large for detecting at anatomical distances, the magnets 40, 50 comprise a material capable of holding a sufficient magnetic strength, such as ferromagnetic materials, particularly nickel, iron, cobalt, or rare earth magnetic materials. Moreover, the size of the magnets is larger than 0.1 mm³. It is also preferable to avoid using magnets having too great a size, because larger sized magnetized particles make the absolute position of the magnet more difficult to determine. Thus, the magnets in an exemplary preferred embodiment have a size of less than 1 cm³. The optimal size for the magnets will depend on the geometry of the implant 10, the distance of the magnets 40, 50 from the sensors 30, and other considerations. For orthopedic implants, the magnets 40, 50 are preferably between about 0.5 mm and 2.5 mm in diameter and essentially spherical.

The magnetic field produced by a uniformly magnetized spherical magnet composed of homogenous material is accurately expressed in Cartesian coordinates by the following equation:

$${\vec{H}}_{dip} ( x , y , z ) = \frac{1}{4 π {\sqrt{{x}^{2} + {y}^{2} + {z}^{2}}}^{5}} [ \begin{matrix}- 2 {x}^{2} + {y}^{2} + {z}^{2} & - 3 xy & - 3 xz \\ - 3 xy & {x}^{2} - 2 {y}^{2} + {z}^{2} & - 3 yz \\ - 3 xz & - 3 yz & {x}^{2} + {y}^{2} - 2 {z}^{2}\end{matrix} ] [ \begin{matrix}{m}_{x} \\ {m}_{y} \\ {m}_{z}\end{matrix} ] \qquad ( eq . 1 )$$

where x, y, and z are coordinates corresponding to the position vector r, which are unknown, and mₓ, m_{y}, and m_{z} are the orthogonal components of the magnetic moment vector m, which are known. In general, the magnetic field produced by real magnetic materials is not perfectly spherical, nor homogenous, nor uniformly magnetized. This would lead to deviations from the theoretical field of equation 1, however the real magnetic fields can be reasonably estimated by taking into account the higher order multipole terms. The potential function used to calculate higher order multipole terms can be derived from the following equation:

$$ϕ = {\sum }_{n = 0}^{∞} {ϕ}_{n} = {\sum }_{n = 0}^{∞} \frac{{( - 1 )}^{n}}{4 π} \frac{{ρ}^{( n )}}{n !} \frac{{\partial }^{n}}{\partial {r}^{n}} ( \frac{1}{r} ) \qquad eq . ( 2 )$$

where r is the distance from the source and is the moment.

In an exemplary embodiment of the invention, an array of n sensors 30ᵢ (I=1−n) is positioned surrounding the implant 10. This array of sensors 30ᵢ₋ₙ is then used to determine the location of a plurality of magnets 40,50. The minimum number of sensors 30 requires is equal to the number of degrees of freedom for each magnet being measured multiplied by the number of magnets. Thus, for six magnets with three degrees of freedom (x, y, and z positional coordinates, assuming known magnetic moment orientation), at least 18 single-axis sensors 30 are required to provide a definitive solution. In a preferred exemplary embodiment, a sufficient number of sensors 30 are provided to over-determine the positions of the magnets to provide greater accuracy and resolution. For example, in the knee joint implant described above, an array of at least 20 sensors is provided.

The method used to determine the positions of the magnets 40, 50 may be the well-known least squares scanning approach, although other methods, such as the Multiple Signal Classification (MUSIC) may also be utilized. The basic problem assumes that the implant 10 contains M magnetic dipoles of fixed orientation, but at unknown locations. Each sensor observes a field, B_{obs}(i) with index I corresponding to the I^{th} sensor among N total sensors. The searching procedure comprises the steps of shifting the hypothetical dipoles around in a 3-dimensional grid, calculating the expected magnetic field B_{dip} of each sensor for the assumed position of the hypothetical dipoles, and comparing the expected fields to the measured fields. The positions of the magnets 40, 50 being the hypothetical dipole locations where the expected field best fit the field observed by the sensor array. The measure of fit is defined as the square of the Frobenius norm, which is given by:

$$F = {\sum }_{i = 1}^{N} {‖ {\sum }_{j = 1}^{M} {{B}^{->}}_{dip} ( {{S}^{->}}_{i} - {{R}^{->}}_{j} ) - {{B}^{->}}_{obs} ( i ) ‖}_{F}^{2} \qquad ( eq . 3 )$$

where Sᵢ denoted the location of the i^{th} sensor in the array, and Rⱼ denotes the position of the j^{th} dipole. In essence, the dipole locations are determined by finding values of R that minimze F.

In an exemplary embodiment of the invention, the magnets 40, 50 are arranged in a pre-determined spatial relationship to each other and/or to the implant 10. Affixing the magnets 40, 50 in a pre-determined spatial relationship relative to the implant 10 allows the precise position of the entire implant to be determined by locating the magnets. Moreover, micro-motion between components of the implant can be monitored. Affixing the magnets 40, 50 to the implant 10 with a pre-determined special relationship to each other reduces the quantity of iterations necessary to locate each magnet.

I claim:

1. An imaging system for locating a discrete number of magnets with predetermined magnetic field maps fixed in a medical implant, comprising:
at least one magnetic field sensor arranged in an array around the implant; and
a computational component configured to calculate the locations of the magnets by iteratively solving for position vector coordinates of the magnets using the pre-determined magnetic field maps of the magnets and the magnetic field measurements observed by each sensor.

2. The imaging system of claim 1, wherein the magnetic field map of each magnet is approximated by multipole expansion.

3. The imaging system of claim 1, wherein the magnetic field map of each magnet is approximated by integrating over the equivalent magnetic monopole distribution within the magnet's volume.

4. The imaging system of claim 1, wherein the magnetic field map of each magnet is approximated by integrating the assumed magnetization distribution within the magnet's volume.

5. The imaging system of claim 1, wherein the locations of the magnets are determined using a least squares analysis, in which the expected field due to a hypothetical distribution of magnets produces the closest representation of the field observed by all the sensors.

6. The imaging system of claim 1, wherein the locations of the magnets are determined using a maximum likelihood estimation analysis.

7. The imaging system of claim 5, wherein the locations of the magnets are determined using a multiple signal classification approach.

8. The imaging system of claim 1, wherein a pre-determined spatial relationship between some of the magnets with respect to other magnets is used to reduce the quantity of iterations.

9. The imaging system of claim 1, further comprising a graphics component configured to convert the position vectors of the dipoles into a viewable image of the implant.

10. The imaging system of claim 1, wherein the magnets have a number of degrees of freedom, and the array of sensors comprises a quantity of sensors that is at least as great as the number of degrees of freedom for the magnets.

11. The imaging system of claim 1, wherein the magnets comprise ferromagnetic material.

12. The imaging system of claim 5, wherein the magnets are spheres.

13. The imaging system of claim 12, wherein the magnets have a diameter of between about 0.1 mm and 2.5 mm.

14. A method of imaging an implant, comprising the steps of:
affixing a pre-determined quantity of magnets with pre-determined field maps to the implant;
disposing an array of magnetic field sensors around the implant; and
iteratively solving for the position vectors of the magnets using the pre-determined field maps for the magnets and the magnetic field measurements observed by each sensor; and
using the determined set of magnet locations to infer the relative position of at least one component in the implant with respect to another component.

15. The method of claim 14, wherein the locations of the magnets are determined using a least squares test to compare the expected field for each of a progression of theoretic magnet position sets to the magnetic fields measured by the sensors.

16. The method of claim 14, wherein the locations of the magnets are determined using a multiple signal classification approach.

17. The method of claim 14, wherein the locations of the magnets are determined using a maximum likelihood estimation method.

18. The method of claim 14, wherein a pre-determined spatial relationship between the magnets is correlated to reduce the quantity of iterations.

19. The method of claim 14, further comprising the step of forming a viewable image of the implant using the position vectors of the magnets.

20. An imaging system, comprising:
an array of magnets fixed in an implant and having pre-determined quantity, magnetic field map, and moment orientations; and
an array of sensors arranged around the implant;
wherein the locations of the magnets are determined by iteratively solving for the position vector coordinates using the pre-determined quantity, magnetic field map, and moment orientations and the magnetic field measurements for each sensor.

21. The imaging system of claim 20, wherein the magnets comprise ferromagnetic material.

22. The imaging system of claim 21, wherein the magnets are uniformly magnetized spheres.

23. The imaging system of claim 22, wherein the magnets have a diameter of between about 0.1 mm and 2.5 mm.

24. An implant comprising a pre-determined quantity of magnets fixed thereto for visualization of the implant using static magnetic fields, the magnets having a pre-determined magnetic field map and pre-determined moment orientations.

25. The implant of claim 24, wherein the implant comprises at least two structural components, and each component has three magnets affixed thereto.

26. The implant of claim 24, wherein the magnets are affixed in pre-determined locations relative to each other and the implant.

27. The implant of claim24, wherein the magnets comprise ferromagnetic material.

28. The implant of claim 27, wherein the magnets are spheres or ellipsoids.

29. The implant of claim 28, wherein the magnets have a diameter of between about 0.1 mm and 2.5 mm.
